(19) United States
(12) Patent Application Publication
(10) Pub. No.: US 2023/0398081 A1
(43) Pub. Date: Dec. 14, 2023

(54) ADDITIVE MANUFACTURING METHOD FOR DRUG DELIVERY DEVICES

(71) Applicants: DOSER IP B.V., Leiden (NL); ACADEMISCH ZIEKENHUIS LEIDEN H.O.D.N. LUMC, Leiden (NL)

(72) Inventors: Niels OUWERKERK, Leiderdorp (NL); Kirsten Johanna Maria SCHIMMEL, Leiden (NL)

(73) Assignees: DOSER IP B.V., Leiden (NL); ACADEMISCH ZIEKENHUIS LEIDEN H.O.D.N. LUMC, Leiden (NL)

(21) Appl. No.: 18/249,833

(22) PCT Filed: Oct. 22, 2021
(86) PCT No.: PCT/NL2021/050640

§ 371 Date: Apr. 20, 2023

(30) Foreign Application Priority Data
Oct. 22, 2020 (NL) 2026725

(51) Int. Cl. A61K 9/20; A61K 9/24; A61K 31/635; A61K 31/519; B33Y 10/00; B33Y 80/00

(52) U.S. Cl. CPC A61K 9/2095; A61K 9/209; A61K 31/635; A61K 31/519; A61K 9/2013; B33Y 10/00; B33Y 80/00

(57) ABSTRACT

The invention relates to an extrusion additive manufacturing method for forming a drug delivery device. The method includes providing a first drug delivery material containing a first major excipient with a first melting range and a first melting peak. The method further includes extruding the first delivery material at an extrusion temperature that is within the melting range of the first major excipient.

The invention is in the field of additive manufacturing methods for forming drug delivery devices. The invention is in particular directed to an extrusion additive manufacturing method for forming drug delivery devices. The invention further relates to a cartridge and a system for use in the method.

At the moment more than 50% of the medications are provided in solid dosage forms including tablets. The manufacturing of such solid dosage forms is however often complex, as it may require a plurality of ingredients and processing steps. Accordingly, it is costly and cumbersome to produce small batches. As such, the customizability of the medication is limited, and the available dosages are dictated by the suppliers. Patients and healthcare professionals may thus be restricted in their choices. Customizability of drugs has recently gained more interest as it allows for the specific adjustment of several parameters (e.g. drug concentration and release profile) to fit a patient's needs. Customized medication may be provided by additive manufacturing (also referred to as 3D printing). This allows for a high level of control over several aspects such as drug release rate, dissolution, drug concentration, shape and volume of the formulation. Additionally, on-demand drugs (i.e. drugs that are manufactured when the need arises) are desired as this limits the need for a long shelf life and stability of the drugs. Concomitantly, on-demand formulation of these drugs is also desirable.

One type of additive manufacturing for drugs is powder-bed fusion. The FDA has approved one drug to be manufactured via this method (Spritam®). The precise printing has provided the drug with the ability to disintegrate instantly after ingestion. However, powder-bed fusion additive manufacturing of drugs is still limited as the powder beds typically comprise fine powders. These fine powders are difficult to handle due to, for instance, agglomeration. Additionally, cleaning may be cumbersome and lots of the powder bed remains as waste after the production of the drugs.

U.S. Pat. No. 9,381,154 discloses an alternative additive manufacturing method for forming one or more layers of a drug delivery device by ejecting one or more components though one or more nozzles. This method does not require a powder bed. The components are dissolved or dispersed in a pharmaceutically compatible phase change ink. The obtained drug delivery devices may have a variety of adjustable properties such as drug release profile and drug concentration. However, the APIs are often difficult to dissolve in the ink and therefore typically only low dose drugs are manufactured. Additionally, due to the fluidity it is a challenge to obtain a reproducible shape and there is often a lack of control over the deposition.

Another possibility of additive manufacturing for drugs is extrusion additive manufacturing. Extrusion additive manufacturing is a method of 3D printing that may be used for a variety of materials such as thermoplastics, metals and ceramics. EP3482774 describes an extrusion additive manufacturing method for solid self-emulsifying systems comprising an active ingredient and excipients that comprises a lipophilic constituent and/or a surfactant. Herein, the printing is performed at a temperature higher than the melting temperature of the mixture of the excipients, preferably 15° C. above the melting temperature. This i.a. has the drawback of poor control over the deposition and concomitantly a poor reproducibility. Moreover, a high process temperature may lead to partial decomposition of the API.

When the feedstock for extrusion additive manufacturing comprises filaments it is typically referred to as fused filament fabrication. An example hereof is disclosed in WO2016/038356. It discloses a method using filaments comprising the active ingredient. It is further disclosed that the method provides dose-customizable solid dosage forms on-demand and increased storage stability. However, the use of filaments is complex as they tend to break on the spool and typically have high melting points and thus require high extrusion temperatures. Additionally, the drug release is often slow and difficult to adjust.

It is an object of the present inventors to provide an improved extrusion additive manufacturing method for forming a drug delivery device that at least partially overcomes the above-mentioned drawbacks. The inventors found an extrusion additive manufacturing method that allows for drug delivery devices to be produced at relatively low temperatures.

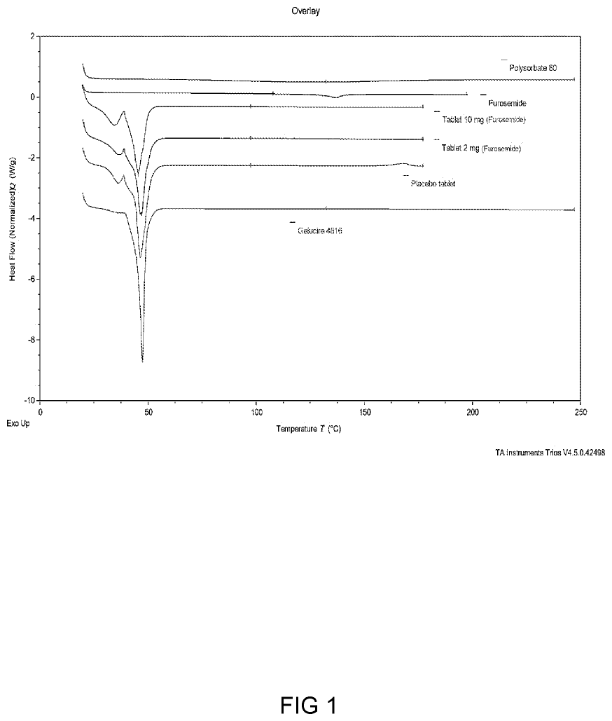

FIG. 1 illustrates DSC curves of several constituents and drug delivery devices according to the invention.

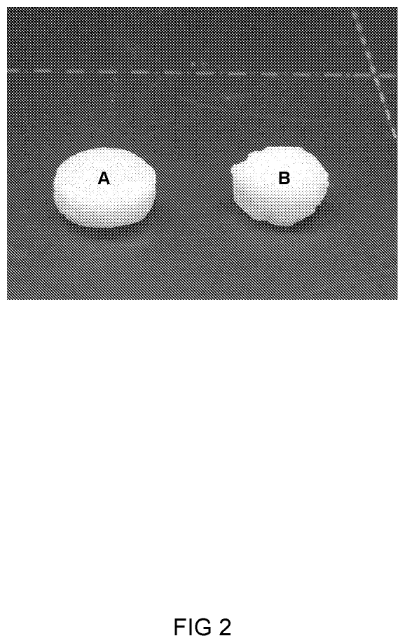

FIG. 2 illustrates examples of tablets printed at different temperatures.

Thus, in a first aspect the invention is directed to an extrusion additive manufacturing method for forming a drug delivery device comprising:
providing a first drug delivery material comprising a first major pharmaceutically acceptable excipient that exhibits a first melting range comprising a first peak temperature;
extruding the first drug delivery material at an extrusion temperature; wherein the extrusion temperature is within the melting range of the first major excipient.

The drug delivery devices produced via the method according to the present invention are typically solid and preferably tablets. The solid dosage forms are preferred due to the ease of administration resulting in increased patient compliance. Additionally, solid dosage forms may have a longer shelf life. The drug delivery devices are preferably suitable for oral and/or rectal administration, more preferably for oral administration.

A first drug delivery material comprising a first major excipient that exhibits a first melting range that comprises a first peak temperature is provided. Herein the term ‘first melting range’ refers to the temperature range from a first initial melting temperature until a first final melting temperature. The melting range and its peak temperature can be determined by conventional methods such as differential scanning calorimetry (DSC). At room temperature (approximately 20° C.) the first major excipient is typically semi-solid (e.g. a wax) or solid. The excipient starts to melt at the first initial melting temperature and continues to melt until the first final melting temperature has been reached. The first final melting temperature is typically the temperature at which the melting appears to have ended.

Additionally, the first melting range comprises a first peak temperature. The first peak temperature is the temperature at the maximum or minimum of the thermal event. In general, only one peak temperature occurs in a melting range. This temperature is often approximately in the middle of the melting range.

The DSC curve that may be obtained for the first major excipient usually depicts one or more (e.g. sharp) bell-shaped curves. An example hereof can be seen in FIG. 1 labeled as Gelucire 4816. The first bell-shaped curve for an endothermic process is referred to as the ‘first melting range’, where the temperature at which a maximum of energy input is reached (if endothermic, as typically visualized on the y-axis) is defined as the peak temperature (Tₚₑₐₖ). After this peak temperature the curve thus declines and may return to values similar to the (e.g. extrapolated) base line. The first initial temperature (Tᵢₙᵢₜᵢₐₗ) is the temperature at which the curve deviates from the (optionally extrapolated) base line towards the peak temperature. The first final temperature (T_{final}) is similarly the temperature at which the curve returns to the (e.g. extrapolated) base line after the peak temperature has been reached.

It may be that the first major excipient comprises one or more impurities or additives that affect the melting range (e.g. broadening and/or lowering). Additionally, the first major excipient may have a second melting range. For instance, it may be possible to have solid particles remaining in the molten first major excipient after the first melting range. These particles typically melt in a second melting range starting at a higher initial melting temperature. Alternatively or additionally, a phase change (e.g. crystallization) may be observed for which a second bell-shaped curve is obtained. This curve typically corresponds to an exothermic process and therefore not referred to as the melting range. This phase change curve may, for instance, be visible before the first melting range. Additionally, dependent on the stability of the first major excipient and/or the storage conditions, the phase change curve may increase, decrease or remain equal after a certain storage time. Nonetheless, it is typically the case that the DSC curve of the major excipient shows only one thermal event corresponding to the first melting range. In cases the drug delivery device comprises further excipients and/or one or more active pharmaceutical ingredients, the DSC curve of a drug delivery device, as seen in FIG. 1 for instance for Tablet 2 mg (Furosemide) and Tablet 10 mg (Furosemide) typically illustrates a more complex curve.

The first drug delivery material comprises a first major pharmaceutically acceptable excipient (herein referred to as excipient). The excipient is generally not pharmaceutically active and may be considered as a carrier material for the formulation and delivery of a pharmaceutical active ingredient, which may also be present in the drug delivery device. The first major excipient is typically present in an amount larger than the other constituents individually. Moreover, the first major excipient is further typically present in more than 50 wt % of the total content of the drug delivery device. Excipients are used in the pharmaceutical industry for e.g. stabilization or therapeutic enhancement. Additionally, the excipient may facilitate drug absorption, reduce viscosity and/or enhance the solubility of an active ingredient. The excipient is typically selected for being safe for administration, for example they may be on the generally recognized as safe (GRAS) list. The GRAS list is established by the Food and Drug Administration (FDA) of the United States.

Preferably the first major excipient comprises polyoxylglycerides, fatty alcohols, fatty acids, hard fats, one or more polyethylene glycol (PEG) mono- and/or diesters with fatty acids and optionally one or more mono, di- and/or triglycerides, or a combination thereof. A typical suitable fatty alcohol and/or fatty acid comprises a C₈-C₁₈ aliphatic tail. Preferably, the first major excipient comprises PEG32 mono- and/or diesters with C₈-C₁₈ fatty acids. C₈-C₁₈ is used herein to indicate that the fatty acid comprises a carbon chain of 8 to 18 carbon atoms. The number average molecular weight of PEG32 is typically around 1500 g/mol. Advantageously, the first major excipient is typically stable and inert towards other constituents in the first drug delivery material. Longer PEG chains may be used to increase the melting point, while shorter PEG chains are typically used to lower the melting point. Lowering the melting point may be preferred for dosage forms such as suppositories, that typically require a melting point of approximately 37° C. (body temperature).

Additionally, the first major excipient is typically a semi-solid waxy material and may have an amphiphilic character. The amphiphilic character can be expressed by a hydrophilic-lipophilic balance (HLB). The HLB is typically determined by the balance of the size and strength of the hydrophilic and lipophilic moieties of a molecule. Herein the HLB is based on Griffin's method that ranges from 0 (completely hydrophobic) to 20 (completely hydrophilic). The HLB may be used to predict the surfactant properties of a molecule. Typically, molecules with an HLB above 10 are considered water-soluble and below 10 are considered lipid-soluble. Molecules with an HLB value from approximately 16 to 18 may be used as solubilizer or hydrotrope.

In a particular embodiment of the present invention, the first major excipient comprises one or more polyoxylglycerides.

Polyoxylglycerides are mixtures of monoesters, diesters, and triesters of glycerol, and monoesters and diesters of polyethylene glycols (PEG); see also Rowe et al., Handbook of Pharmaceutical Excipients, 6^{th} Edition, p. 557 et seq. Polyoxylglycerides, also referred to as macrogolglycerides, can be used as self-emulsifying and solubilizing agents in oral pharmaceutics. Certain polyoxylglycerides are known and commercially available as Gelucire®. Certain Gelucire® mixtures such as Gelucire® 48/16 however lack the esters of glycerol that are usually present in polyoxylglycerides.

Polyoxylglycerides suitable for the present invention can be prepared by partial alcoholysis of glycerides with PEG. Accordingly, in a typical embodiment, the ester(s) of glycerol and the ester(s) of PEG in the major excipient may be based on the same fatty acid(s), e.g. one or more of C₈-C₁₈ fatty acids.

In principle any polyoxylglycerides can be used for the present invention. Polyoxylglycerides can be classified (see e.g. Handbook Pharmaceutical Excipients, 6^{th} edition, page 557-560 and V. Jannin et al. International Journal of Pharmaceutics 466 (2014) 109-121) into caprylocaproyl polyoxylglycerides (decanoic acid, mixed with monoesters with glycerol and actanoic acid; poly(oxy-1,2-ethanecliyl), α-hydro-ω-hydroxy-, mixed decanoate and octanoate), lauroyl polyoxylglycerides (lauric acid, diester with glycerol; poly(oxy-1,2-ethanediyl), α-(1-oxododecyl)-ω-[(1-oxododecyl)oxy]), linoleoyl polyoxylglycerides (corn oil, ethoxylated; 9,12-octadecadienoic acid (9E,12E)-monoester with 1,2,3-propanetriol), oleoyl polyoxylglycerides (9-octadecenoic acid (9Z)-, monoester with 1,2,3-propanetriol; poly(oxy-1,2-ethanediyl), α-[(9Z)-1-oxo-9-octadecenyl]-G3-hydroxy) and stearoyl polyoxylglycerides (distearic acid, diester with glycerol; poly(oxy-1,2-ethanecliyl), α-(1-oxooctadecyl)-ω-[(1-oxooctadecyl)-oxy]). Accordingly, the excipient preferably comprises a polyoxylglyceride that is selected from the group consisting of: caprylocaproyl polyoxylglycerides, lauroyl polyoxylglycerides, linoleoyl polyoxylglycerides, oleoyl polyoxylglycerides, stearoyl polyoxylglycerides and combinations thereof.

Particular good results were obtained when the excipient comprises the preferred polyoxylglyceride, and accordingly especially when the excipient comprises one or more of mono, di and triglycerides with PEG esters, which are therefore most preferred.

A particularly preferred the first major excipient is commercially available as Gelucire® (e.g. Gelucire® 48/16, see also K. C. Panigrahi et al. Future Journal of Pharmaceutical Sciences 4 (2018) 102-108). The first number following the name Gelucire® (e.g. 50/*, 44/*, 48/*, 55/*, etc.) indicates the melting point in degrees centigrade while the second number (e.g. */13, */14, */16, */18, etc.) indicates the HLB hydrophilicity. In this respect, the commercially available Gelucire® 48/16 is preferred for the present invention.

Hard fats (also referred to a suppository bases, hydrolyzed fat or hard fat triglyceride esters) are mainly based on mixtures of the triglyceride esters of the higher saturated fatty acids (C₈H₁₇COOH to C₁₈H₃₇COOH) along with varying proportions of mono- and diglycerides (see also Handbook Pharmaceutical Excipients, 6^{th} edition, page 722-726). Hard fats can be used for suppositories because they typically have a melting point between 27 and 45° C. such as about 33 to 34° C. This makes the hard fats also very suitable as a major excipient for the present invention. Commercially available hard fats that are for example suitable include those available under the tradename Witepsol® which have a peak melting point in the range of about 30 to about 40° C.

Conventionally, it is typically used to increase the bioavailability of poorly-water soluble drugs. The bioavailability may relate to i.a. parameters such as solvability, membrane permeability and enzymatic degradation of the drug in the patient. Additionally, the dissolution of drugs is typically an important factor for the bioavailability. Dissolution may relate to the extent and rate of the transfer of the individual drug molecules from the solid to a solution. Typical dissolution tests include the Paddle dissolution test and are typically known to a person skilled in the art.

Optionally, the first drug delivery material comprises a plasticizer. A plasticizer is typically responsible for decreasing the plasticity and/or viscosity of a material and to enhance the thermal stability. Often the plasticizers do not form chemical bonds and fulfill their function through intermolecular forces. Suitable plasticizers may include glycerol and/or polysorbate, such as polysorbate 80. Glycerol may also be present in the major excipient (vide supra), but an additional portion of glycerol may have further beneficial effects due to its plasticizing ability. Additionally or alternatively, it may be preferred for the first drug delivery material to comprise one or more additives such as sweeteners, color agents, dyes, flavors and/or pigments. The additives can be beneficial e.g. for patient compliance.

The first drug delivery material may further comprise an active pharmaceutical ingredient (API). The API may also slightly function as a plasticizer, thereby possibly aiding the extrusion of the drug delivery material according to the present invention. The terms “API”, “drug”, “active ingredient” are used interchangeably herein. As it is not required that the first drug delivery material comprises an API it is possible to produce a drug delivery device with no drugs present (i.e. a placebo). An example of the DSC curve of a placebo tablet can be seen in FIG. 1. Thus, it may be appreciated that the present invention is also very suitably for the preparation of placebo drugs. This is particularly advantageous for clinical trial applications, wherein relatively small amounts of formulations and placebos are required. The excipient in the first drug delivery material typically enhances the solubility of the API and moreover, may enhance the bioavailability of the API.

Before filling the cartridge with the API and the excipient, typically, the API and excipient are mixed and heated to a slightly elevated temperature. The slightly elevated temperature is preferably sufficient to obtain a melt of the excipient. After mixing, the mixture is typically weighed and added to a cartridge (vide infra). The weighing may be done when the mixture is cold, or it may be done when the mixture is still at the slightly elevated temperature. Additionally, air may be excluded during the weighing.

Most APIs are poorly water-soluble and are typically defined as such if the highest dosage strength is not soluble in 250 ml of an aqueous media over a pH range of 1 to 7.5. Thus, preferably a solubilizing environment for the API is created. This can be provided by an excipient with an HLB above 10, preferably above 15. Accordingly, it is preferred that the API is an apolar API. Nonetheless, it is possible to use a suspension of the API in the excipient for forming a drug delivery device according to the present invention. Therefore, other APIs such as polar APIs and/or ionized APIs that may be combined with their counter ion (e.g. as a Na or HCl salt) can also be used. For example, sildenafil citrate is a suitable API salt for the present invention. A list of possible APIs is mentioned in Pharmacopeia. The effective dose of a specific API is typically known to a person skilled in the art.

In order to form the drug delivery device, the first drug delivery material is extruded at an extrusion temperature that is within the melting range of the first major excipient. Preferred major excipients often have relatively low melting ranges and low peak temperatures that allow for easy processing. Accordingly, the first drug delivery material comprises at least one excipient that has a first peak temperature between 30-60° C., preferably between 40-50° C.

It is further preferred to have the extrusion temperature below the first peak temperature. Preferably the extrusion temperature is within the first 75%, more preferably within the first 50% of the temperature range between the first initial melting temperature and the first peak temperature. The first initial melting temperature is thus included in the temperature range. Accordingly, the extrusion temperature is preferably between 30-60° C., more preferably between 30-45° C., most preferably between 42-43° C.

This extrusion temperature is typically sufficient for lowering the viscosity of the first drug delivery material but also to maintain a sufficiently high viscosity for good control over the depositing of the material in e.g. a defined shape. It typically allows for a sufficient surface tension to extrude the material in a preferred shape. Additionally, the temperature typically does not compromise the functionality of the optional API and may allow for easy processing. Furthermore, the cooling period of the deposited material may be minimal resulting in faster production times and limited complexity when depositing a plurality of layers.

It is typically desired that each of the drug delivery devices that are prepared have a similar shape, volume and size (e.g. within 10% difference). This may be beneficial for the further processing of the drug delivery devices such as packaging.

Advantageously, the method preferably comprises providing at least a second drug delivery material that is different from the first drug delivery material. The second drug delivery material may comprise the API in a different concentration than in the first drug delivery material. This enables the provision of a plurality of drug delivery devices that individually comprise a different API concentration throughout the device. By providing a gradient in API concentration or other device characteristics, the release profile of the API can be controlled. The second drug delivery material can thus have any API concentration (0% and up) and can be used as a filler and/or as an additional API source.

The combination of the first and second drug delivery material may accordingly also allow for individual drug delivery systems to be manufactured with an individually different API concentrations but a similar volume, shape and size. As such, the API release profile can be controlled for each individual device that is prepared. This can be used to easily prepare different devices for different patients, but also for different devices for a single patient to facilitate a particular dosage regime.

If the second drug delivery material comprises an API, this may be the same or a different API as present in the first drug delivery material. By including different API's in each drug delivery material, therapeutic treatments involving two API's can be facilitated by providing the opportunity to vary the ratios of the various APIs.

The second drug delivery material comprises a major excipient that is preferably the first major excipient, i.e. the same major excipient as present in the first drug delivery material. However, the major excipient that is present in the second drug delivery material may also be a different major excipient, i.e. a second major excipient. In case the first and the second major excipients are different, it is preferred that they at least have an overlapping melting range, more preferably an overlap before the respective melting peak temperatures, such that they allow extrusion of the first and second drug delivery materials at approximately the same temperature. This prevents or limits the requirement to change the extrusion temperature during the process. Alternatively or additionally, the second drug delivery material may comprise a drug release retardant such as glyceryl behanate and/or a drug release accelerant, such as disintegrants (e.g. crosscarmellose sodium, sodium starch glycolate) to influence the release profile of the API. Drug release retardants and/or drug release accelerants may be used to release the API immediately in e.g. the stomach or may be used to release the API over a longer period of time. It may also be used to allow the API to be mostly released (e.g. over 90%) in the intestines instead of the stomach.

It may be appreciated that the method may comprise any amount of further drug delivery materials that differ from the first drug delivery material. These further drug delivery materials may each differ from each other in terms of one or more APIs, excipients, and the like.

In a preferred embodiment, the method comprises providing at least a third and a fourth drug delivery material, wherein the third drug delivery material comprises a drug release retardant and the fourth drug delivery material comprises a drug release accelerant. The combination of multiple drug delivery materials facilitates the creation of a drug delivery device that provides the optimal drug release profile, as well as reproducible shapes, volumes and sizes.

As for the second drug delivery material, the third, fourth and optionally further drug delivery materials each comprise a major excipient that is preferably the first major excipient, i.e. the same major excipient as present in the first drug delivery material. However, it may be appreciated that the major excipient that is present in the third, fourth and/or further drug delivery materials may also be a different major excipient, i.e. a third, fourth and/or further major excipient. Again, in case the various major excipients are different, it is preferred that they at least have an overlapping melting range, more preferably an overlap before the respective melting peak temperatures, such that they allow extrusion of the different drug delivery materials at approximately the same temperature. This prevents or limits the requirement to change the extrusion temperature during the process.

It may be appreciated that the second, third, fourth and/or further major excipients can be selected from the same group as the first excipient may and may each independently have the same properties as described herein for the first major excipient.

The extrusion process is typically suitable for a continuous flow process with limited start and/or stop commands. A small delay is often observed between executing the start and/or stop command and the actual starting and/or stopping of the process. The pressure switch between printing and not-printing is generally a rapid continuously increasing or decreasing process instead of abrupt. Therefore, typically a small amount of drug delivery material is extruded after giving the stop command, and it typically takes a small amount of time before the drug delivery material gets extruded after the start command has been given. This may result in non-reproducibility of the shapes, volumes and possible API dosage. To increase the reproducibility, the deposition of the drug delivery material of an additional drug delivery device may be started at the position where the printing of the drug delivery material for the previous drug delivery device has ended. Typically, tablet shapes and the like (e.g. oval, capsule, square, almond, doughnut) are produced. In certain embodiments, capsule shapes may be preferred. For quicker release profiles of the API (relative to e.g. oval tablets) doughnut shapes are typically preferred. In certain embodiments, it may be preferred to print tablets by printing double rows as this may advantageously result in a consistent weight distribution which typically allows for better reproducibility. For instance, a relative standard deviation of less than 2%, such as less than 1.3% can be obtained by printing double rows.

Additionally, within each drug delivery device, the printing of the additional layer may be started at the same site as the previously deposited layer. Moreover, the printing of the previously deposited layer preferably ends at the same site as the start of the additional layer. Therefore, preferably a pattern is printed that allows for the start and the end of deposition of each layer to be at the same site. Advantageously, the preferred printing may allow for a maximal drying time of the deposited layer. As the previously deposited layer is typically dried and solidified before the additional layer is printed it may result in a non-compromised drug delivery device. This preferred printing method can advantageously limit the amount of start and/or stop commands. Structures wherein only the inside of the final structure comprises the API are also possible, this may be preferred to retard dissolution. For these structures it may be favorable to print the layers in non-identical patterns. Alternatively or additionally, a nozzle-valve (vide infra) may be present to allow for a more abrupt stop and/or start of the extrusion process that typically allows for an increased control over the printing process of multiple layers and/or multiple drug delivery systems.

Furthermore, the thickness of the layers is a typical important relevant parameter for the drug delivery device. The thickness may i.a. be controlled by the printing or extrusion rate. Additionally, it is typically preferred for the layers to be levelled (i.e. equally thick at all points), for instance with a 10%, preferably 5%, such as 2%, maximum deviation. This may especially be required for the first deposited layer. Advantageously, printing level layers typically results in reproducible and predictable shapes.

Additionally, the absolute thickness of the layers may be a relevant parameter. For example, the first layer may be 0.30-0.60 mm thick, preferably 0.40-0.50 mm such as 0.45 mm while the following layers are 0.30-0.60 mm thick, preferably 0.40-0.50 mm such as 0.43 mm. The following layers are typically slightly pressed in order to adhere better to allow for a stable drug delivery device to be produced. Due to the pressure, the thickness may be minimally reduced. The first layer is preferably not slightly pressed as this may complicate the drug delivery device to be separated from e.g. the holder (vide infra).

The drying and solidifying of the drug delivery material may be enhanced by active cooling after extrusion (e.g. after deposition). The drug delivery material is preferably cooled by a cooled environment in which the extrusion occurs. The environment may for instance be cooled by supplying a cold stream of air. Additionally or alternatively, it may be cooled by a cooled holder that is suitable for receiving the drug delivery device such as a substrate. The holder may be cooled e.g. by an active cooling such as a continuous water flow through the holder.

For easy filling and replacement of at least the first drug delivery material it is preferably provided in a cartridge. More preferably, each drug delivery material is provided in a separate cartridge. Hence, it is preferred to have a cartridge suitable for use in the method according to the present invention that comprises a drug delivery material as described herein above. The cartridge typically comprises a barrel, a piston and an extrusion nozzle. It may for instance be a cylindrical shaped cartridge such as a syringe. The piston is typically used to provide pressure onto the drug delivery material in the barrel in order to extrude the material through the extrusion nozzle. It may therefore be required that the part of the piston that is in contact with the barrel is in direct contact (i.e. no gas flow is possible). The piston may accordingly also be a plunger.

The positioning of the extrusion nozzle is preferably closely monitored. Additionally, the extrusion nozzle preferably has a small opening, for instance a diameter between 0.1 mm and 1 mm, such as 0.4 mm, for the material to be extruded from. This typically allows for good control and a precise deposition of the drug delivery material. It can moreover prevent the extrusion nozzle from clogging, which may be particularly beneficial when printing a suspension of for example the API in the excipient.

Furthermore, the pressure in the cartridge during printing is a typically relevant factor for the reproducibility. Dependent on the stage of the process, the pressure may be kept constant or it may be decreasing or increasing. The pressure generally depends on several factors such as the viscosity of the drug delivery material and/or the opening of the extrusion nozzle. The pressure may accordingly determine the extrusion rate of the drug delivery material. The pressure may be controlled and monitored directly or as a derivative of the force with respect to the area. Herein, the force typically relates to the applied force on the piston and the area to the surface area of the piston that is in contact with the drug delivery material.

Additionally, it is preferred that the cartridge is adapted for individually controlling the temperature of at least the barrel, the piston and/or the extrusion nozzle. The temperature may be controlled at for instance 0.5° C., preferably 0.2° C., accuracy and may thus be slightly different for the individual components of the cartridge. In order to have good control over the temperature, a thermodynamic system may be set up to provide control over the extrusion. This may i.a. include heating the barrel and the material in the barrel of the cartridge to a higher temperature than the nozzle in order to allow fine-tuning the temperature of the extruding material in the nozzle. Accordingly, the barrel, the piston and/or the extrusion nozzle are preferably individually homogeneously heated. The individual and/or homogeneous temperature is typically favorable as this allows for increased control over the process and thus over the final drug delivery device (i.a. the shape). Accordingly, materials with sufficient thermal conductivity are preferably used for the cartridge.

Therefore, it is preferred that the barrel, the piston and/or the extrusion nozzle comprise a metal, or at least their surfaces contacting the drug delivery material consist essentially of metal. More preferably the barrel, the piston and/or the extrusion nozzle are based on metal and may for instance be constructed from stainless steel and/or aluminum, preferably stainless steel.

The cartridge is provided with the drug delivery material loaded therein. Most or all drug delivery material is contained in the barrel. It may be possible for the cartridge to be filled with the drug delivery material if the piston can be taken from the cartridge for example in a screw or cap like manner. Typically, it is beneficial to fill the cartridge with little to no entrapped gas. The gas reduces the space of the cartridge that can be occupied by the drug delivery material and may compromise the control over the deposition. Additionally, the gas may compromise the stability of the drug delivery material during storage. To ensure the gas can be released during and/or after the filling it is preferred that the barrel and/or the piston comprise an air outlet. Additionally, the air outlet preferably comprises an air-valve that may be closed to remain gas-tight after the entrapped gas is released.

As describe herein above it may be preferred to have abrupt stop and/or start processes as this allows for improved control and better reproducibility. Therefore, it is preferred that the extrusion nozzle comprises a nozzle-valve.

The barrel, piston and/or extrusion nozzle and concomitantly the drug delivery material are typically pre-heated before extrusion. Heating of the drug delivery material typically results in a small expansion of the material and may result in an increased pressure in the cartridge. This may increase to the extent that the drug delivery material leaks from the cartridge. The preferred nozzle-valve typically prevents this leakage. Furthermore, the air outlet may also be beneficial to prevent leakage as the built-up pressure within the cartridge may be released.

Additionally, the drug delivery devices are pharmaceuticals that should be safe for administration to the patient. Therefore, it may be preferred that the drug delivery devices and/or the cartridges are manufactured under good manufacturing practice (GMP). Moreover, the filling of the cartridges may also be done under GMP.

A further aspect of the invention is directed to a drug delivery device obtainable by the method according to the present invention. The drug delivery device may thus be formed by consecutive printing of layers. These layers are typically printed in the pattern and according to the process as previously described. Therefore, the drug delivery device typically comprises a plurality of layers. Within the device, some of its layers typically differ in their respective compositions, based the presence and amount of the first, second, third and/or fourth drug delivery material. For example, each layer may comprise the drug delivery materials at another concentration. As the additional layer is typically printed directly on the previous layer the individual layers may not be structurally visual. However, the layers may for example comprise additives that cause a change in appearance (e.g. color) to the extent that the layers can be visually detected. Additionally or alternatively, the layers may be chemically different as for instance a first layer may comprise the first drug delivery material and a second layer may comprise the second drug delivery material. The plurality of layers can be beneficial for the customizability of the drug delivery device as the layers may be used to form a gradient of the API and/or any of the additives, such as drug release or drug retardants over the drug delivery device. The gradient is typically a non-continuous gradient as there is typically no gradient present within one layer.

Another aspect of the invention is directed to a system comprising a three-dimensional printing apparatus for extrusion additive manufacturing according to the method of the present invention. The system comprises one or more cartridge holders suitable for holding one or more cartridges as described herein above. The multiple holders may individually be used for the first, second, third and/or fourth drug delivery material. Additionally, the system comprises a holder for receiving the drug delivery device. The holder for receiving the drug delivery device may for instance be a substrate. Additionally, the holder for receiving the drug delivery device may be cooled (e.g. by a continuous water flow through the holder). Alternatively, the deposited material may be cooled by the environment. Nonetheless, the cooling typically ensures a sufficient cooling rate for the deposited drug delivery material in order maintain control over the shape and volume of the drug delivery device.

The invention can be illustrated with the following non-limiting examples.

### Example 1—Placebo

Syringe Preparation

Approximately 3-5 grams of Gelucire® 48/16 is weighted and introduced in a syringe of suitable dimensions and made of suitable material and equipped with a nozzle suitable for printing. The syringe is fitted with a plunger that is pressed until the solid provides back pressure. The assembly is heated to melt the contents in an inverted position and any remaining air is pressed out. The assembly is left to cool. After cooling the assembly can be stored.

Printing

For printing the syringe is mounted on a custom 3D printer and the syringe and nozzle are heated to 43° C. and 42° C. respectively. Once at the necessary temperature, the printing is initiated and 20 circular shapes of 7 layers are printed.

Result

The weight of the shapes was on average 125 mg with less than 4% relative standard deviation.

### Example 2—Furosemide

Syringe Preparation

Furosemide (a typical DSC curve thereof can be seen in FIG. 1) is accurately weighted and mixed with a sufficient amount of excipient (Gelucire® 48/16) to make an 8 wt % solution. The mixture is stirred while heating to melt the material. About 3-5 grams is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 43° C. and 42° C. respectively. Once at the necessary temperature, the printing is initiated and 20 circular shapes of 7 layers are printed.

Result

The content of furosemide was 10 mg with less than 2% relative standard deviation.

Dissolution Test

Dissolution testing of the dose forms shows that more than 80% of the API is released within 60 minutes or quicker. The dissolution test was performed by a Paddle dissolution test in an aqueous HCl solution at a pH of 1.2.

### Example 3—Sildenafil

Syringe Preparation

Sildenafil is accurately weighted and mixed with a sufficient amount of excipient (Gelucire® 48/16) to make about a 3.3 wt % dispersion. The mixture is stirred while heating to melt the material. About 3-5 grams is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 43° C. and 42° C. respectively. Once at the necessary temperature, the printing is initiated and 20 circular shapes of 7 layers are printed.

Result

The content of Sildenafil was 4 mg with less than 3% relative standard deviation.

### Example 4—Furosemide 2 mg with Plasticizer

Syringe Preparation

2 mg of furosemide per tablet is accurately weighted and mixed with a sufficient amount of excipient (Gelucire® 48/16) and polysorbate 80. The mixture is heated to melt at 50° C. and stirred to obtain a homogeneous mixture. The mixture is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 41° C. Once at the necessary temperature, the printing is initiated and 7 layers per tablet were printed. The tablets were printed in double rows of ach 12 tablets, to obtain 24 tablets per batch.

Result

The content of furosemide was 2 mg with less than 3.5% relative standard deviation.

Dissolution Test

Dissolution testing of the dose forms shows that on average more than 75% of the API is released within 45 minutes or quicker. The dissolution test was performed by a Paddle dissolution test in a phosphate buffer solution of pH 5.8.

### Example 5—Furosemide 10 mg with Plasticizer

Syringe Preparation

10 mg of furosemide per tablet is accurately weighted and mixed with a sufficient amount of excipient (Gelucire® 48/16) and polysorbate 80. The mixture is heated to melt at 50° C. and stirred to obtain a homogeneous mixture. The mixture is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 41° C. Once at the necessary temperature, the printing is initiated and 7 layers per tablet were printed. The tablets were printed in double rows of ach 12 tablets, to obtain 24 tablets per batch.

Result

The content of furosemide was 2 mg with approximately 2% or less relative standard deviation.

Dissolution Test

Dissolution testing of the dose forms shows that more than 80% of the API is released within 45 minutes or quicker. The dissolution test was performed by a Paddle dissolution test in a phosphate buffer solution of pH 5.8.

### Example 6—Sildenafil 4 mg with Plasticizer

Syringe Preparation

4 mg of sildenafil per tablet is accurately weighted and mixed with a sufficient amount of excipient (Gelucire® 48/16) and polysorbate 80. The mixture is heated to melt at around 50-55° C. until the mixture is homogeneous. The mixture is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 44° C. and 41° C., respectively. Once at the necessary temperature, the printing is initiated and 7 layers per tablet were printed. The tablets were printed in double rows of ach 12 tablets, to obtain 24 tablets per batch.

Result

The content of sildenafil was 4 mg with approximately less than 3% relative standard deviation.

Dissolution Test

Dissolution testing of the dose forms shows that more than 80% of the API is released within 45 minutes or quicker. The dissolution test was performed by a Paddle dissolution test in an aqueous HCl solution at a pH of 2.0.

### Example 7—Witepsol Placebo

Syringe Preparation

Approximately 3-5 grams of Witepsol is heated to melt at around 40-45° C. until the mixture is homogeneous. The mixture is introduced in a syringe of suitable dimensions and made of suitable material and equipped with a nozzle suitable for printing. The syringe is fitted with a plunger and air is pressed out. The assembly is left to cool down. After cooling the assembly can be stored.

Printing

For printing the syringe is mounted on a custom 3D printer and the syringe and nozzle are heated to 30° C. and 30° C. respectively. Once at the necessary temperature, the printing is initiated and 4 suppository shapes are printed.

Result

The weight of the shapes was on average 650 mg with less than 2% relative standard deviation.

### Example 8—Placebo Printed at 43° C. (Syringe) and 42° C. (Nozzle)

Syringe Preparation

Approximately 3-5 grams of Gelucire® 48/16 is weighted and introduced in a syringe of suitable dimensions and made of suitable material and equipped with a nozzle suitable for printing. The syringe is fitted with a plunger that is pressed until the solid provides back pressure. The assembly is heated to melt the contents in an inverted position and any remaining air is pressed out. The assembly is left to cool. After cooling the assembly can be stored.

Printing

For printing the syringe is mounted on a custom 3D printer and the syringe and nozzle are heated to 43° C. and 42° C. respectively. Once at the necessary temperature, the printing is initiated and 20 circular shapes of 7 layers are printed.

Result

The tablets have a regular shape with clearly visible printlines, as visualized in FIG. 2 (tablet A). The weight of the shapes was on average 125 mg with less than 4% relative standard deviation.

### Example 9—Placebo Printed at 45° C. (Syringe and Nozzle)

Syringe Preparation

Approximately 3-5 grams of Gelucire® 48/16 is weighted and introduced in a syringe of suitable dimensions and made of suitable material and equipped with a nozzle suitable for printing. The syringe is fitted with a plunger that is pressed until the solid provides back pressure. The assembly is heated to melt the contents in an inverted position and any remaining air is pressed out. The assembly is left to cool. After cooling the assembly can be stored.

Printing

For printing the syringe is mounted on a custom 3D printer and the syringe and nozzle are heated to 45° C. and 45° C. respectively. Once at the necessary temperature, the printing is initiated and 20 circular shapes of 7 layers are printed.

Result

The tablets are irregularly shaped as illustrated in FIG. 2 (tablet B).

### Example 10—Placebo Printed at 48° C. (Syringe and Nozzle)

Syringe Preparation

Approximately 3-5 grams of Gelucire® 48/16 is weighted and introduced in a syringe of suitable dimensions and made of suitable material and equipped with a nozzle suitable for printing. The syringe is fitted with a plunger that is pressed until the solid provides back pressure. The assembly is heated to melt the contents in an inverted position and any remaining air is pressed out. The assembly is left to cool. After cooling the assembly can be stored.

Printing

For printing the syringe is mounted on a custom 3D printer and the syringe and nozzle are heated to 48° C. and 48° C. respectively. Once at the necessary temperature, the printing is initiated and 20 circular shapes of 7 layers are printed.

Result

The resulting shapes have a very poor appearance. The viscosity is too low for a neat tablet to be printed.

1. An extrusion additive manufacturing method for forming a drug delivery device comprising:
providing a first drug delivery material comprising a first major pharmaceutically acceptable excipient, which first major pharmaceutically acceptable excipient exhibits a first melting range comprising a first peak temperature;
extruding the first drug delivery material at an extrusion temperature; wherein the extrusion temperature is within the melting range of the first major excipient.

2. The method according to claim 1, wherein the first major excipient comprises fatty alcohols, fatty acids, hard fats, one or more polyethylene glycol (PEG) mono- and/or diesters with fatty acids and optionally one or more mono, di and/or triglycerides or a combination thereof, preferably the first major excipient comprises PEG32 mono- and/or diesters with C₈-C₁₈ fatty acids, more preferably the first major excipient comprises a polyoxylglyceride, most preferably selected from the group consisting of: caprylocaproyl polyoxylglycerides, lauroyl polyoxylglycerides, linoleoyl polyoxylglycerides, oleoyl polyoxylglycerides, stearoyl polyoxylglycerides and combinations thereof, more preferably a lauroyl polyoxylglyceride, most preferably said excipient comprises one or more of mono, di and triglycerides with PEG esters.

3. The method according to claim 1, wherein the first major excipient has a first peak temperature between 30-60° C., preferably between 40-50° C. and/or wherein said excipient has a hydrophilic-lipophilic balance (HLB) above 10, preferably above 15.

4. The method according to claim 1, wherein the first drug delivery material further comprises an active pharmaceutical ingredient (API), preferably an apolar API.

5. The method according to claim 1, wherein the extrusion temperature is lower than the first peak temperature, preferably within the first 75%, more preferably within the first 50% of the temperature range between the first initial melting temperature and the first peak temperature, preferably between 30-60° C., more preferably between 30-45° C., most preferably between 42-43° C.

6. The method according to claim 1, wherein the first drug delivery material comprises the active pharmaceutical ingredient (API) and wherein the method further comprises providing at least a second drug delivery material that is different from said first drug delivery material, preferably, wherein said second drug delivery material comprises the API in a different concentration than said first drug delivery material.

7. The method according to claim 6, wherein said second drug delivery material comprises a drug release retardant and/or a drug release accelerant.

8. The method according to claim 6, wherein the method further comprises providing at least a third and a fourth drug delivery material, wherein the third drug delivery material comprises a drug release retardant and the fourth drug delivery material comprises a drug release accelerant.

9. The method according to claim 8, wherein the first, second, third and/or fourth drug delivery material after extrusion are deposited in individual layers that are essentially equally thick, preferably with a 10%, more preferably 5%, such as 2%, maximum relative deviation in thickness.

10. The method according to claim 9 wherein the first, second, third and/or fourth drug delivery material after extrusion are deposited in individual layers, wherein a first layer is 0.30-0.60 mm thick, preferably 0.40-0.50 mm such as 0.45 mm and following layers are 0.30-0.60 mm thick, preferably 0.40-0.50 mm such as 0.43 mm.

11. The method according to claim 8 wherein the method further comprises cooling the first, second, third and/or fourth drug delivery material after extrusion and deposition, preferably by a cooled environment and/or by a cooled holder suitable for receiving the drug delivery device.

12. The method according to claim 1, wherein the first drug delivery material, preferably each drug delivery material is provided in a cartridge.

13. A drug delivery device obtainable by the method according to claim 1.

14. The drug delivery device according to claim 13, comprising a plurality of layers, which at least two of these layers differ in their respective compositions.

15.-18. (canceled)
